(19) United States
(12) Patent Application Publication
(10) Pub. No.: US 2021/0301308 A1
(43) Pub. Date: Sep. 30, 2021

(54) SYSTEMS AND METHODS FOR APTAMER-BASED INTRACELLULAR DELIVERY OF A PAYLOAD USING NANONEEDLES

(71) Applicants: MEKONOS INC., San Francisco, CA (US); THE BOARD OF TRUSTEES OF THE LELAND STANFORD JUNIOR UNIVERSITY, Stanford, CA (US)

(72) Inventors: Anil NARASIMHA, Stanford, CA (US); Carolyn BERTOZZI, Stanford, CA (US); Arunava Steven BANERJEE, San Francisco, CA (US)

(21) Appl. No.: 17/266,275

(22) PCT Filed: Aug. 5, 2019
(86) PCT No.: PCT/US2019/045130

§ 371 Date: Feb. 5, 2021

Related U.S. Application Data
(60) Provisional application No. 62/715,074, filed on Aug. 6, 2018.

(51) Int. Cl. C12N 15/89; C12N 15/115

(52) U.S. Cl. CPC C12N 15/89; C12N 2320/32; C12N 2310/16; C12N 15/115

(57) ABSTRACT

Methods, chips and systems are provided for the ex vivo delivery of payloads into cells using an aptamer-based approach. Methods are provided for delivering a payload to a cell comprising providing a nanoneedle and a polynucleotide, wherein a first end of the polynucleotide comprises an aptamer capable of binding a molecule endogenous to a cell, wherein the first end of the polynucleotide is conjugated to the nanoneedle, and wherein a second end of the polynucleotide comprises an oligonucleotide capable of hybridizing with a payload; contacting the payload with the polynucleotide, wherein the payload contains a nucleotide sequence that is complementary to the oligonucleotide sequence; and inserting the nanoneedle into a cell, wherein upon insertion of the nanoneedle into the cell the payload is released from the polynucleotide.

### RELATED APPLICATIONS

The present application claims priority to and the benefit of, U.S. Provisional Patent Application No. 62/715,074, titled “APTAMER BASED INTRACELLULAR DELIVERY OF A PAYLOAD USING NANONEEDLES” and filed on Aug. 6, 2018, the entire contents of which are hereby incorporated by reference for all purposes.

### FIELD

The embodiments disclosed herein are generally directed towards aptamer-based systems and methods of delivering a payload to a cell. More specifically, there is a need for a universal platform for delivering any type of payload to any type of cell.

### BACKGROUND

Despite its essential role in biological research and therapeutic applications, the efficient intracellular delivery of exogenous compounds and macromolecular cargo remains a long-standing challenge. The limitations of established delivery technologies have hampered progress in multiple areas as the potential of exciting new materials, insights into disease mechanism, and approaches to cell therapy are not fully realized owing to their delivery hurdles. This challenge can be viewed through the lens of, for example, two broad parameters: cell type and target material. Existing technologies are mainly focused on addressing a subset of combinations, specifically nucleic acid delivery (that is, transfection) to immortalized cell lines and certain primary cells. Some of the most exciting target cell types, such as stem cells and immune cells, are also the most difficult to address. Versatile and robust methods of delivering almost any cargo molecule to any cell type are much needed.

As such, there is a need for a universal delivery system and method as described herein.

### SUMMARY

In one aspect, a method of delivering a payload to a cell is provided, the method comprising providing a nanoneedle and a polynucleotide, wherein a first end of the polynucleotide comprises an aptamer capable of binding a molecule endogenous to a cell. Further, the first end of the polynucleotide is conjugated to the nanoneedle, and a second end of the polynucleotide comprises an oligonucleotide capable of hybridizing with a payload. The method further comprises contacting the payload with the polynucleotide, wherein the payload contains a nucleotide sequence that is complementary to the oligonucleotide sequence. The method further comprises inserting the nanoneedle into a cell, wherein upon insertion of the nanoneedle into the cell the payload is released from the polynucleotide.

In another aspect, a chip for delivering a payload to a cell is provided, the chip comprising a solid support, and a nanoneedle attached to the solid support and configured to receive a polynucleotide. A first end of the polynucleotide comprises an aptamer capable of binding a molecule endogenous to a cell. The first end of the polynucleotide is capable of conjugating to the nanoneedle. A second end of the polynucleotide comprises an oligonucleotide capable of hybridizing with one of a plurality of molecules of a payload.

In yet another aspect, a system for delivering a payload to a cell is provided, the system comprising a plurality of nanoneedles, and a plurality of polynucleotides, wherein a first end of a respective polynucleotide of a respective one of the plurality of polynucleotides comprises an aptamer capable of binding a molecule endogenous to the cell. The first end of the respective polynucleotide is conjugated to one of the plurality of nanoneedles. A second end of the respective polynucleotide comprises an oligonucleotide capable of hybridizing with one of a plurality of molecules of a payload. The system further comprises a plurality of wells and an injecting device that houses the plurality of nanoneedles thereon, the injecting device configured to move the plurality of nanoneedles to within the plurality of wells.

Additional aspects will be evident from the detailed description that follows, as well as the claims appended hereto and the drawings.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIGS. 1A-1D illustrates a method of delivering a payload to a cell, in accordance with various embodiments.

FIG. 2 illustrates a chip for delivering a payload to a cell, in accordance with various embodiments.

FIG. 3 illustrates a system for delivering a payload to a cell, in accordance with various embodiments.

It is to be understood that the figures are not necessarily drawn to scale, nor are the objects in the figures necessarily drawn to scale in relationship to one another. The figures are depictions that are intended only to bring clarity and understanding to various embodiments of the methods, apparatuses, and systems disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Various embodiments, features, objects, and advantages of the present teachings will be apparent from the description and accompanying drawings, and from the claims. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, a tray, a baseplate, a separate metal structure, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

The term “nucleic acids” as used herein may include any polymer or oligomer (oligonucleotide) of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). The present disclosure contemplates any deoxyribonucleotide (DNA), ribonucleotide (RNA) or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “aptamer” as used herein refers to an oligonucleotide that is capable of forming a complex with an intended target substance.

The term “nanoneedle” as used herein can refer to an object that acts as a penetration device to deliver a payload. The payload can be delivered, for example, via an outer surface of the object or through a flow passage of the object. The object will typically be in the nanometer size range. The object can be solid in construction with the object possessing any contemplated geometry. The object can have, for example and not limited to, a conical, tubular, square, rectangular, triangular, pentagonal, hexagonal, or oval shape, though any shape that allows for payload delivery is contemplated herein. The object can also, or alternatively, possess a flow passage for which the payload will pass through. The flow passage can possess any contemplated geometry. The flow passage can have, for example and not limited to, a conical, tubular, square, rectangular, triangular, pentagonal, hexagonal, or oval shape, though any shape that allows for payload delivery is contemplated herein.

Unless otherwise defined, scientific and technical terms used in connection with the present teachings described herein shall have the meanings that are commonly understood by those of ordinary skill in the art.

The present disclosure relates to a universal platform which enables precise ex vivo delivery of any size and type of payloads into target cells. The disclosure provides a method of delivering a payload to a cell, which utilizes an array of controllable nanoneedles that are conjugated to a polynucleotide which contains both an aptamer capable of binding to a molecule found inside the cell; and an oligonucleotide capable of recognizing an aptamer.

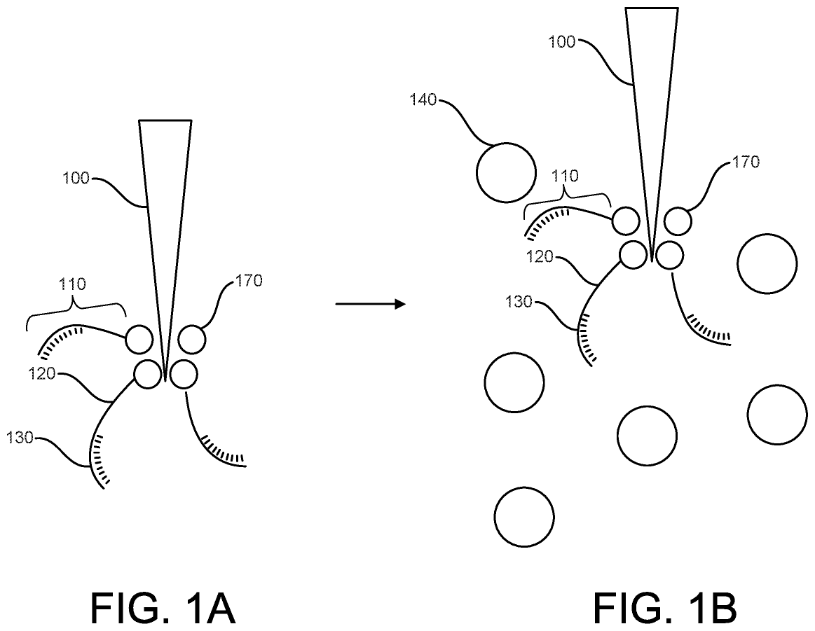

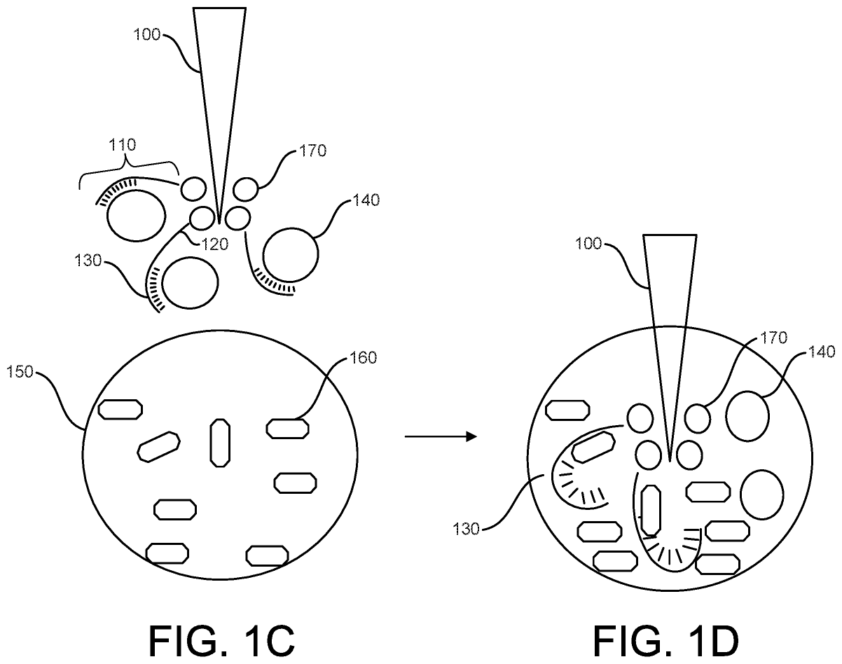

In accordance with various embodiments, a method of delivering a payload to a cell is provided as illustrated, for example, in FIGS. 1a to 1d. As provided in FIG. 1a, a nanoneedle 100 and a polynucleotide 110 can be provided, wherein a first end of the polynucleotide comprises an aptamer 120 capable of binding a molecule 160 (see FIGS. 1c-d) endogenous to a cell 150 (see FIGS. 1c-d). The first end of the polynucleotide can be conjugated to the nanoneedle 100 through attachment to a binding particle 170 that is coated on the nanoneedle 100 and a second end of the polynucleotide can comprise an oligonucleotide 130 capable of hybridizing with a payload 140. As provided in FIG. 1b, the method can comprise the step of contacting the payload 140 with the polynucleotide 110, wherein the payload 140 can include a nucleotide sequence that is complementary to the oligonucleotide 130. As provided by FIGS. 1c-d, the method can also comprise the step of inserting the nanoneedle 100 into cell 150 wherein upon insertion of the nanoneedle 100 into the cell 150 the payload 140 is released from the polynucleotide 110 and delivered to the cell 150.

Further methods will be discussed below, as will be embodiments related to chips and systems. In all of the preceding and succeeding embodiments of methods, chips and systems provided herein, all features discussed herein related to, for example, the nanoneedle and associated materials, well features and well types, chip features and chip types, polynucleotides and corresponding attachment and coupling features, nanoneedle coatings, aptamers, oligonucleotides, payload and payload types and payload delivery techniques to target (e.g., cell), cell features and cell type, scalable systems, and agricultural applications, apply to any and all embodiments described herein (including, for example, any and all methods, chips and systems embodiments provided herein).

In accordance with various embodiments (e.g., method, chip and system embodiments) provided herein, the tip of the nanoneedle generally can be sized to deliver a payload to the cell or to a particular organelle within the cell (e.g., the nucleus). In various embodiments, the tip of the nanoneedle can have a diameter between about 10 nm to 200 nm. In various embodiments, a diameter of about 50 nm can be sufficient to deliver a payload to both the cell cytoplasm and to the cell nucleus for most all cell types. In various embodiments, the diameter of the nanoneedle may depend in part on the size of the target cell or organelle. In various embodiments, the diameter is less than 1 μm.

In accordance with various embodiments (e.g., method, chip and system embodiments) provided herein, more specific diameters for the nanoneedle can include about 10 nm, about 15 nm, about 16 nm, about 17 nm, about 18 nm, about 19 nm, about 20 nm, about 25 nm, about 30 nm, about 35 nm, about 40 nm, about 45 nm, about 50 nm, about 55 nm, about 60 nm, about 65 nm, about 70 nm, about 75 nm about 80 nm, about 85 nm about 90 nm about 95 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 600 nm, about 650 nm, about 700 nm, about 750 nm, about 800 nm, about 850 nm, about 900 nm, about 950 nm, about 1 μm, or ranges between any two of these values.

In accordance with various embodiments (e.g., method, chip and system embodiments) provided herein, the nanoneedle can be used to deliver a payload to a specific compartment within a cell (e.g., the nucleus, the mitochondria among others). The diameter and length of the nanoneedle may vary for many factors including, for example, the relative size of the cell type and organelle to be targeted. For example, induced pluripotent stem cells (iPS cells) and similar cell types have approximately a diameter range of about 6-10 μm. Other cell types may have larger or smaller dimensions, and thus have correspondingly different size nanoneedles as discussed in detail above.

In accordance with various embodiments (e.g., method, chip and system embodiments) provided herein, more specific lengths for the nanoneedle can include about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm about 16 μm, about 17 μm, about 18 μm, about 19 μm, about 20 μm, about 25 μm, about 30 μm, about 35 μm, about 40 μm, about 45 μm, about 50 μm, about 60 μm, about 70 μm, about 80 μm, about 90 μm, about 100 μm, or a between any of these two values.

In accordance with various embodiments (e.g., method, chip and system embodiments) provided herein, the nanoneedle can be comprised of silicon. The silicon nanoneedle may be coated with various substances to aid conjugation of the polynucleotide to the nanoneedle. In various embodiments, the nanoneedle is coated with gold atoms. Gold nanoparticle coating is useful for attaching biological molecules due to its unique surface, chemical inertness, high electron density and strong optical absorption.

In accordance with various embodiments (e.g., method, chip and system embodiments) provided herein, the nanoneedle may be coated with or comprised of other materials suitable to allow conjugation of nucleic acids to the nanoneedle. For example, the nanoneedle may comprise a Langmuir-Bodgett film, functionalized glass, germanium, PTFE, polystyrene, gallium arsenide, silver, membrane, nylon, PVP, silicon oxide, metal oxide, ceramics or any other material known in the art that is capable of having functional groups such as, for example, amino, carboxyl, Diels-Alder reactants, thiol or hydroxyl incorporated on its surface. Such materials allow the attachment of the nucleic acids and their interaction with target molecules without hindrance from the nanoneedle.

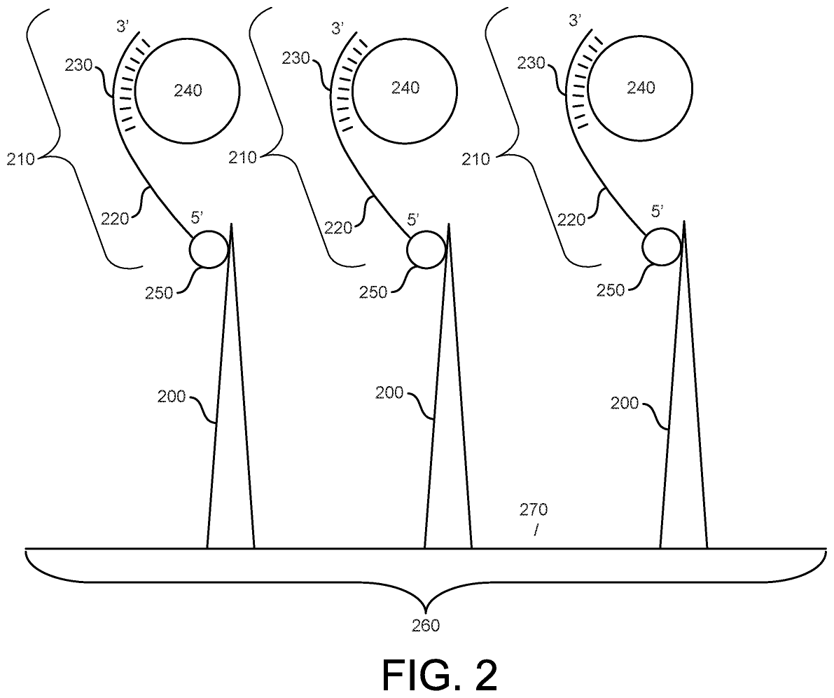

In accordance with various embodiments (e.g., method, chip and system embodiments) provided herein, the polynucleotide can be attached to the nanoneedle in a variety of ways. In various embodiments, the polynucleotide is conjugated to the nanoneedle through a covalent bond. In various embodiments, a thiol (SH) modifier is attached to an end of the polynucleotide. The thiol (SH) modifier enables covalent attachment of the polynucleotide to a variety of surfaces. A SH-modifier can be placed at either the 5′ end or the 3′ end of the polynucleotide (see FIG. 2 for relative locations of 5′ and 3′ ends on polynucleotide 110/210). The SH-modifier allows for covalent linkage with a variety of surfaces including gold nanoparticles.

In accordance with various embodiments (e.g., method, chip and system embodiments) provided herein, the polynucleotide can be coupled to the nanoneedle using avidin/biotin coupling chemistry. In various embodiments, avidin can be immobilized to the nanoneedle via electrostatic interactions (the nanoneedle can be comprised of a negatively charged material such as silicon) and then complex biotinylated nucleotides can be immobilized to the immobilized avidin. In various embodiments, biotinylated polynucleotides are prepared by coupling biotinamidocaproate N-hydroxysuccinimide ester (BCHS) to a modified polynucleotide that contained a 5′ amino group. The BCHS derivative can be used in order to provide a six carbon spacer between the 5′ end of the polynucleotide and the biotin moiety. The spacer is thought to provide the oligonucleotide with more conformational flexibility in hybridizing with other nucleotides. Avidin can be immobilized to silica surfaces by, for example, physical adsorption.

In accordance with various embodiments (e.g., method, chip and system embodiments) provided herein, the polynucleotide may be attached to the nanoneedle through a mediating linker molecule. The mediator linker may be used to connect the aptamer to the binding particle on the nanoneedle. For example, the mediator linker may be polyethylene glycol (PEG) or polyethylenimine (PEI). In accordance with various embodiments, the PEG or PEI linker is thiolated such that it can be used as a linker to connect the polynucleotide to a gold nanoparticle coating the surface of the nanoneedle. The mediator linker can function to decrease nonspecific interactions and increase the biocompatibility and stability of the conjugate. Mediating linkers such as PEG and PEI may also increase hydrophilicity of the payload. In various embodiments, mediating linkers such as PEG or PEI may be used for very hydrophobic payloads that are not easily soluble or payloads that are very irregularly shaped.

In accordance with various embodiments (e.g., method, chip and system embodiments) provided herein, the nanoneedle may be coated with a binding particle (for example, a gold nanoparticle) as shown in FIGS. 1a-d (binding particle 170). In various embodiments, gold-coated nanoneedles can also be conjugated to antibodies. Physical and chemical interactions are used for attaching antibodies to gold atoms. Physical interaction between the antibody and gold atoms can depend on various phenomena including, for example: (a) ionic attraction between the negatively charged gold and the positively charged antibody, (b) hydrophobic attraction between the antibody and the gold surface, and (c) dative binding between the gold conducting electrons and amino acid sulfur atoms of the antibody. Chemical interactions between antibodies and gold nanoparticles can be achieved in a number of ways such as, for example, (i) chemisorption via thiol derivatives, (ii) through use of bifunctional linkers, and (ii) through the use of adapter molecules like Streptavidin and biotin. Conjugation of antibodies can involve covalent and/or non-covalent methods.

As discussed above in reference to FIGS. 1a-1d, polynucleotide 110 can be comprised of at least one aptamer 120 and at least one oligonucleotide 130. The aptamer and the oligonucleotide can be arranged in any order to be conjugated to a nanoneedle 100. In various embodiments, the 5′ end of the aptamer can be conjugated to nanoneedle 100, and the 3′ end of the aptamer is attached to the 5′ end of the oligonucleotide (see, for example, polynucleotide 210 of FIG. 2). In accordance with various embodiments, the 3′ end of the aptamer can be conjugated to the nanoneedle and the 5′ end of the aptamer is fused to the 3′ end of the oligonucleotide. In various embodiments, the oligonucleotide can be conjugated to the nanoneedle at one end and conjugated to the aptamer at the other end. Conjugation can be done in such a way that it does not affect the nucleic acid templates ability to hybridize or subsequent PCR amplification. Such techniques are conventional and well known in the art.

Aptamers, such as those discussed in accordance with various embodiments (e.g., method, chip and system embodiments) provided herein, can be designed to bind to a specific target molecule. Generally, aptamers are macromolecules composed of nucleic acid. As typical of nucleic acids, a particular aptamer may be described by a linear sequence of nucleotides (A, U or T, C and G). The aptamer can be comprised of RNA or DNA. There are no physical limitations to the length of an aptamer. Increasing the length of the aptamer can increase the stability of the aptamer itself. The shape of the aptamer can contribute to its ability to bind tightly against the surface of its target molecule. Since a tremendous range of molecular shapes exist among the possibilities for nucleotide sequences, aptamers may be obtained for a wide array of molecular targets.

In accordance with various embodiments (e.g., method, chip and system embodiments) provided herein, aptamers can be provided that are capable of binding to a molecule that is present in a cell. Such molecules can depend upon the cell type to which the payload is delivered. In various embodiments, the aptamer is capable of binding to adenosine triphosphate (ATP). Because ATP is abundant inside cells, once the payload is delivered to the cell, in various embodiments, ATP will bind to the aptamer, changing the conformation of the polynucleotide, causing the payload to be released into the cell. Other aptamers may be designed which bind to other proteins or molecules found inside the cell as well. For example, in various embodiments, the aptamer recognizes a molecule such as GTP, AKT or Ras. Any molecule present in a concentration large enough in the cell (or target organelle) to compete with the payload for binding on the polynucleotide can be suitable for in accordance with the various embodiments provided herein. In various embodiments, the concentration of the molecule that binds to the aptamer in the cell (or target organelle) can be about 0.01 mM, about 0.5 mM, about 0.1 mM, about 0.2 mM, about 0.4 mM, about 0.6 mM, about 0.8 mM, about 1 mM, about 2 mM, about 3 mM, about 4 mM, about 5 mM, about 6 mM, about 7 mM, about 8 mM, about 9 mM, about 10 mM, about 20 mM, or a range between any two of these values. The relative abundance of the target in the cell and its affinity for the aptamer can affect the efficiency of the release of payload. In various embodiments, mutations can be introduced into the aptamer, which increase or decrease its affinity to a target molecule.

In accordance with various embodiments (e.g., method, chip and system embodiments) provided herein, more than one type of polynucleotide is conjugated to the nanoneedle. In various embodiments, the nanoneedle is capable of delivering more than one payload to a cell at one time. This can be accomplished, for example, by having more than one type of polynucleotide attached to the nanoneedle (i.e. two peptides with unique oligonucleotide sequences). In various embodiments, the two unique polynucleotides contain two unique aptamers and two unique oligonucleotides. In various embodiments, the two unique polynucleotides contain two unique oligonucleotides but the same aptamer. There is no limitation to a particular number of unique polynucleotides to be conjugated to a nanoneedle and is not limited to a particular number of distinct payloads to be delivered to a cell. In various embodiments, about 2, about 3, about 4, about 5, about 6, about 8, or about 10 unique polynucleotide sequences, or a range between any two of these values, is provided.

In accordance with various embodiments (e.g., method, chip and system embodiments) provided herein, the oligonucleotide(s) can also be of variable length. In accordance with various embodiments, the oligonucleotide can be at least 10 nucleotides in length. In accordance with various embodiments, the oligonucleotide can be at least 8 nucleotides (nt) in length. In various embodiments, the length of the oligonucleotide can be about 4 nt, about 5 nt, about 6 nt, about 7 nt, about 8 nt, about 9 nt, about 10 nt, about 11 nt, about 12 nt, about 13 nt, about 14 nt, about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, about 29 nt, about 30 nt, about 35 nt about 40 nt, about 45 nt, about 50 nt, about 55 nt, about 60 nt, about 65 nt, about 70 nt, about 75 nt, about 80 nt, about 85 nt, about 90 nt, about 95 nt, about 100 nt, or a range between any two of these values. The oligonucleotide can be comprised of DNA or RNA. The oligonucleotide does not have to be 100% complementary to a nucleotide sequence found on the payload. In various embodiments, the oligonucleotide may comprise a sufficient number of bases complementary to bases found on the payload such that the payload hybridizes with the polynucleotide with sufficient strength to deliver the payload to the cell. In various embodiments, the percentage of nucleotide bases on the oligonucleotide which correspond to complementary bases on the payload is about 50%, about 55%, about 60%, about 65%, about 70%, about 75%, about 80%, about 85%, about 90%, about 95%, about 100%, or a range between any two of the values.

In accordance with various embodiments (e.g., method, chip and system embodiments) provided herein, the payload can be a nucleotide based molecule. For example, in various embodiments, the payload is comprised of either DNA or RNA such as, for example, a viral DNA or RNA particle. In various embodiments, a portion of payload comprises a sequence complementary to the oligonucleotide such that the payload hybridizes with the oligonucleotide. It is not necessary that the payload be 100% complementary to the oligonucleotide, so long as the payload is capable of hybridizing with the oligonucleotide with sufficient strength to be delivered to a cell. For example, 8-12 nucleotides of the payload are capable of hybridizing with the oligonucleotide. In various embodiments, the payload length can be about 4 nt, about 5 nt, about 6 nt, about 7 nt, about 8 nt, about 9 nt, about 10 nt, about 11 nt, about 12 nt, about 13 nt, about 14 nt, about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, about 29 nt, about 30 nt, about 35 nt about 40 nt, about 45 nt, about 50 nt, about 55 nt, about 60 nt, about 65 nt, about 70 nt, about 75 nt, about 80 nt, about 85 nt, about 90 nt, about 95 nt, or about 100 nt, or a range between any two of these values which are capable of hybridizing (complementary with) the oligonucleotide. The nucleotides of the payload that hybridize with the oligonucleotide may be present anywhere on the nucleotide-based payload (3′ end, 5′ end or anywhere in between), as long as the payload is capable of hybridizing with sufficient strength to be delivered to a cell. In various embodiments, the payload may be a circular nucleotide sequence. In various embodiments, the payload may be a linear nucleotide sequence. In various embodiments, the payload is a single stranded nucleotide. In various embodiments, the payload is circular DNA (i.e. plasmids). The payload can also be linear DNA. The payload can also be a hybrid DNA-RNA molecule.

In accordance with various embodiments (e.g., method, chip and system embodiments) provided herein, any sort of payload to any type of cell can be delivered using the various embodiments provided herein. In various embodiments, proteins, peptides, metabolites, viruses, capsid nanoparticles, membrane impermeable drugs, exogenous organelles, molecular probes, nanodevices and nanoparticles are all potential payloads for intracellular delivery. When such molecules are not “nucleotide-based” (i.e. not comprised of DNA or RNA), the payload may be conjugated to a short nucleotide sequence with bases complementary to the oligonucleotide strand. In various embodiments, the short nucleotide sequence length can be about 4 nt, about 5 nt, about 6 nt, about 7 nt, about 8 nt, about 9 nt, about 10 nt, about 11 nt, about 12 nt, about 13 nt, about 14 nt, about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, about 29 nt, about 30 nt, about 35 nt, about 40 nt, about 45 nt, about 50 nt, about 55 nt, about 60 nt, about 65 nt, about 70 nt, about 75 nt, about 80 nt, about 85 nt, about 90 nt, about 95 nt or about 100 nt, or a range between any two of these values.

In accordance with various embodiments (e.g., method, chip and system embodiments) provided herein, methods, chips and systems are provided for systematic delivery of protein biologics into living cells, such as inhibitory antibodies and stimulatory transcription factors. In various embodiments, methods, chips and systems are provided for silencing DNA. For example, the payload could comprise siRNA, HDAC inhibitors, DNA methyltransferase inhibitors, or other molecules that can have increase or decrease gene expression. In various embodiments, the payload is a protein (such as an antibody or an enzyme) or a small molecule drug capable of inhibiting or enhancing a specific intracellular signaling pathway. In various embodiments, methods, chips and systems are provided for the intracellular delivery of nanodevices, sensors and probes for the measurement of physical and chemical properties within the cell. In various embodiments, the probes can be generated from functional materials, such as nanoplasmonic optical switches, carbon nanotubes and quantum dots. In various embodiments, the methods, chips and systems provided herein may be used to deliver proteins integral for the efficacy of certain functions (e.g. reprogramming of cells into a stem cell). In various embodiments, the payload is a transcription factor such as Oct4 and Sox2. In various embodiments, the payload delivers the transcription factor directly into the nucleus. Such delivery can increase efficiency of reprogramming cells into induced pluripotent stem cells (iPS cells). Delivery of proteins may be, in certain situations, necessary to forego the transcription/translation of proteins that could really limit the efficiency of reprogramming. In various embodiments, the methods, chips and systems provided herein can include directly delivering proteins to cells that have a mutated or non-functional cytoplasmic protein that renders an aberrant phenotype (e.g., adding a functional Ras molecule to a cell type that has a dominant negative Ras).

In accordance with various embodiments (e.g., method, chip and system embodiments) provided herein, the payload comprises genetic material capable of stably integrating into the cell's genome. In various embodiments, CRISPR based technologies can be utilized. For example, in various embodiments, the payload is a gRNA molecule comprised of crRNA and a tracrRNA capable of complexing with a Cas protein (for example, Cas9, Cas 12, Cas13a or any other Cas molecule) inside the cell to mediate cleave of target DNA sites that are complementary to any 20 nucleotides of the gRNA (CRISPR-CAS system). In various embodiments, this gRNA is delivered to a Cas expressing cell. In various embodiments, a Cas expressing cell is first created, for example, by first transfecting the cells with a lentiviral vector expressing Cas or a Cas analogue or derivative. In various embodiments, the Cas can be delivered to the cell, in accordance with various embodiments, wherein Cas protein (or plasmid expressing Cas) is the payload. In various embodiments, the gene for expressing Cas and the gene for expressing gRNA are placed in a single vector. In various embodiments, the Cas gene and the gRNA gene are placed under the control of two different promoters. In accordance with various embodiments, at least two unique polynucleotides are conjugated to the nanoneedle: the first polynucleotide comprising an oligonucleotide capable of hybridizing with the gRNA, and the second polynucleotide comprising an oligonucleotide capable of hybridizing with Cas or a Cas analogue or derivative. In accordance with various embodiments, a third unique polynucleotide can be conjugated to the nanoneedle, which can comprise an oligonucleotide capable of hybridizing with a piece of donor DNA for insertion into the cell's genome using the CRISPR-CAS system.

Other genome-editing technologies may also be delivered to a cell in accordance with various embodiments (e.g., method, chip and system embodiments). For example, transcription activator-like effector nucleases (TALENs) or zinc finger nucleases may be delivered to the cell as a payload. Both TALENs and zinc finger nucleases are proteins that recognize specific DNA sequence and can induce DNA cleavage and subsequent genome editing. Either the TALEN or zinc-finger nuclease can be conjugated to a unique DNA sequence capable of hybridizing with the oligonucleotide. In various embodiments, at least two unique polynucleotides are provided: 1) the first polynucleotide comprising an oligonucleotide capable of hybridizing to a TALEN or zinc-finger nuclease protein for cleaving the cell's genomic DNA at a precise sequence; and 2) the second polynucleotide comprising an oligonucleotide capable of hybridizing to a piece of donor DNA for insertion into the cell's genome. In various embodiments, both polynucleotides can be conjugated to the same nanoneedle.

In accordance with various embodiments (e.g., method, chip and system embodiments) provided herein, transient changes in gene expression may be desirable. For example, in various embodiments, the payload may comprise genetic material that enables ex vivo differentiation for the manufacturing of cell therapies. However, once the desired cell types are created and deployed into patients, expression of the genetic material might no longer be necessary for the therapeutic application, and may even have adverse effects. In such instances, transient changes in gene expression could be appropriate. In various embodiments, the payload can be comprised of genetic material that is transiently expressed in the target cell, but not stably integrated into the cell's genome.

The accordance with various embodiments (e.g., method, chip and system embodiments) provided herein can be applicable to any cell-type. For example, in various embodiments the target cell is an immune cell (e.g., a B-cell or a T-cell). In various embodiments, the target cell is a stem cell (e.g. hematopoietic stem cell, embryonic stem cell, induced pluripotent stem cell). The target cell is not limited to a specific cell type or organism. The cell could be, for example, eukaryotic or prokaryotic; a plant or animal or bacterial cell. The cell types may also be cell lines that have been used for manipulation, such as, for example, HeLa, 293s, cancer/transformed cells, and primary cells (any organ-derived cell type may be used). The cells can also be from model organisms such as, for example, Drosophila, C. elegans, zebrafish, Xenopus, and yeast. The cells can also be from other plant model organisms such as, for example, Arabidopsis.

The various embodiments (e.g., method, chip and system embodiments) provided herein can provide for a scalable system for cell-based therapies. In accordance with various embodiments (e.g., method, chip and system embodiments), methods, chips and systems can be provided for delivering genetic material to self-renewing hematopoietic stem cells and T cells for cancer therapy. In accordance with various embodiments (e.g., method, chip and system embodiments), methods, chips and systems can be used for ex vivo cell-based gene therapies, such as CAR-T cell therapy, immunotherapy, self-renewing hematopoietic stem cells and T cells for immunotherapy. For example, in hematopoietic stem cells, ex vivo gene therapy can be used to correct mutations in monogenic diseases such as combined immunodeficiency (SCID)-X1, Wiskott-Aldrich Syndrome and β-thalassemia. For T cells, novel function against tumor targets can be instructed ex vivo by induced expression of specific T cell receptors and chimeric antigen receptors followed by adoptive cell transfer. In various embodiments, solid tissues may also be manipulated ex vivo using the methods, chips and systems provided herein. For example, the various methods, chips and system embodiments provided herein could be used to facilitate tissue engineering, through the ex vivo transduction of epidermal stem cells, bone, spleen, lung, colon or any other solid tissue cell type. In various embodiments, induced secretion of cytokines or programmed drug resistance and safety switches can be engineered into these cell types by ex vivo manipulation.

The various embodiments (e.g., method, chip and system embodiments) provided herein may also be used for agricultural based methods as well. For example, the various method, chips and systems embodiments provided herein may be used to study molecular mechanisms underlying plant function, fight disease, and enhance plant productivity. Genetic engineering in plants is becoming a fast growing field, where manipulation of the plant's genome can enhance areas like plant breeding, stability of plants, and resistance to harmful viruses/bacteria. CRISPR/Cas systems can be delivered to produce a variety of results, including altering the plant height of the rice plant, as well as introducing a mutation in a gene in the soybean plant to increase vegetative size. Delivering these payloads is sometimes even harder in plant cells as they have a cell wall, which is by definition hard to penetrate. Using a nanoneedle-aptamer based approach, as provided by various embodiments herein, can traverse the cell wall and deliver payloads with higher efficiency.

In various embodiments, a method of delivering a payload to a cell is provided comprising providing a nanoneedle and a polynucleotide. A first end of the polynucleotide can comprise an aptamer capable of binding a molecule endogenous to the cell. The first end of the polynucleotide can be conjugated to the nanoneedle. A second end of the polynucleotide can comprise an oligonucleotide capable of hybridizing with the payload. The method can further comprise orienting the nanoneedle such that it is designed and configured to be placed into contact with a payload. The method can further comprise inserting the nanoneedle into the inner volume of a well that is designed and configured to house a cell wherein the nanoneedle is designed and configured such that, upon insertion of the nanoneedle into the cell, the payload is released from the polynucleotide and delivered to the cell.

In accordance with various embodiments (e.g., method, chip and system embodiments), a well can be provided to hold a cell such that the nanoneedle can be inserted into the cell to deliver a payload. The well can be of any size, shape or material so long as it is capable of housing a single cell. In various embodiments, the well can be coated with a substance (for example, poly-lysine) which allows the cell to adhere to the well. In various embodiments, the well can be a partition. In various embodiments, the well can be a chamber. In various embodiments, the well can be a microfluidic chamber. In various embodiments, the well is a tube. In various embodiments, the well can be a microarray. In various embodiments, the well can be a lane (e.g., a lane on a flow cell). In various embodiments, the well can be a cell-trapping device (e.g., via di-electrophoresis). In various embodiments, a plurality of wells can be arranged on a microplate.

As discussed above, in all of the preceding and succeeding embodiments of methods, chips and systems provided herein, all features discussed herein related to, for example, the nanoneedle and associated materials, well features and well types, chip features and chip types, polynucleotides and corresponding attachment and coupling features, nanoneedle coatings, aptamers, oligonucleotides, payload and payload types and payload delivery techniques to target (e.g., cell), cell features and cell type, scalable systems, and agricultural applications, apply to any and all embodiments described herein (including, for example, any and all methods, chips and systems embodiments provided herein).

In accordance with various embodiments, a chip 260 for delivering a payload to a cell is provided as illustrated for example, by FIG. 2. The chip 260 can comprise a nanoneedle (or a plurality of nanoneedles 200 as illustrated) configured to receive a polynucleotide (or a plurality of polynucleotides 210 as illustrated, wherein each nanoneedle is configured to receive an associated polynucleotide. Moreover, the first end of each polynucleotide can comprise an aptamer 220 capable of binding a molecule endogenous to the cell. Moreover, first end of each polynucleotide 210 can be conjugated to the associated nanoneedle 200. Further, a second end of each polynucleotide can comprise an oligonucleotide 230 capable of hybridizing with a payload 240. In various embodiments, the chip can comprises a single nanoneedle. In various embodiments, the chip 260 can comprise a solid support 270 for which the nanoneedle (or plurality of nanoneedles as illustrated) is attached.

In accordance with various embodiments (e.g., method, chip and system embodiments), the chip can be made of any material capable of providing the solid support 270 on which to attach the needle or plurality of nanoneedles. In various embodiments, the chip is made of silicon. In various embodiments, the chip is made of glass. In various embodiments, the chip is comprised of a polymer. In various embodiments, the chip is comprised or more than one substrate.

In accordance with various embodiments (e.g., method, chip and system embodiments), a plurality of nanoneedles is attached to the chip. In various embodiments, there are about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8900, about 9000, about 9100, about 9200, about 9300, about 9400, about 9500, about 9600, about 9700, about 9800, about, 9900, about 10000, about 1 million, about 2 million, about 3 million, about 4 million or about 5 million nanoneedles, or a range between any two of these values are attached to the chip.

In accordance with various embodiments (e.g., method, chip and system embodiments), the well can be provided to hold a cell such that the nanoneedle can be inserted into the cell to deliver a payload. The well can be of any size, shape or material so long as it is capable of housing a single cell. In various embodiments, the well can be coated with a substance (for example, poly-lysine) which allows the cell to adhere to the well. In various embodiments, the well can be a partition. In various embodiments, the well can be a chamber. In various embodiments, the well can be a microfluidic chamber. In various embodiments, the well is a tube. In various embodiments, the well can be a microarray. In various embodiments, the well can be a lane (e.g., a lane on a flow cell). In various embodiments, the well can be a cell-trapping device (e.g., via di-electrophoresis). In various embodiments, a plurality of wells can be arranged on a microplate.

As discussed above, in all of the preceding and succeeding embodiments of methods, chips and systems provided herein, all features discussed herein related to, for example, the nanoneedle and associated materials, well features and well types, chip features and chip types, polynucleotides and corresponding attachment and coupling features, nanoneedle coatings, aptamers, oligonucleotides, payload and payload types and payload delivery techniques to target (e.g., cell), cell features and cell type, scalable systems, and agricultural applications, apply to any and all embodiments described herein (including, for example, any and all methods, chips and systems embodiments provided herein).

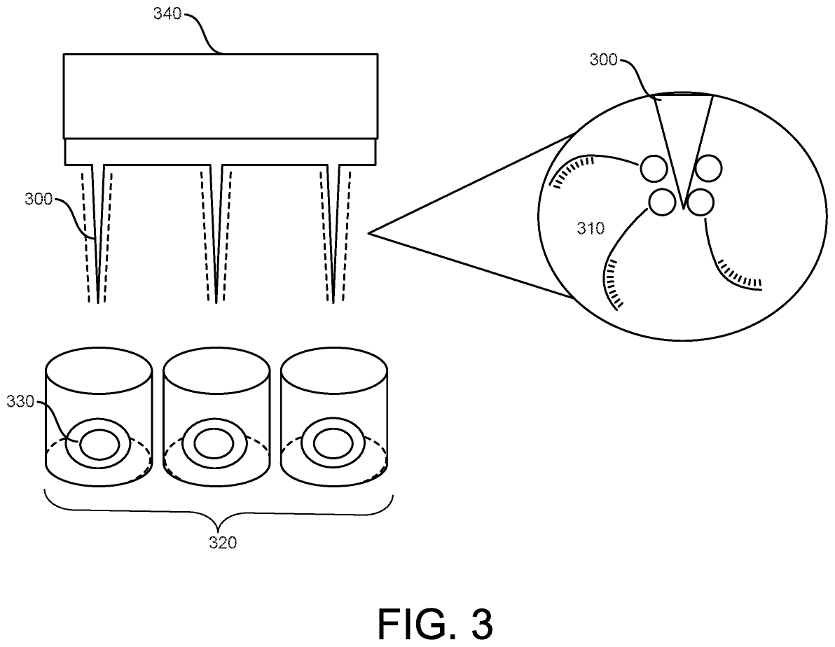

In accordance with various embodiment, a system for delivering a payload to a cell is provided as illustrated in FIG. 3. The system can comprise a plurality of nanoneedles 300, and a plurality of polynucleotides 310. A first end of a respective polynucleotide can comprise an aptamer capable of binding a molecule endogenous to the cell (see discussion above related to FIGS. 1a-1d). The first end of the respective polynucleotide can be conjugated to an associated one of the plurality nanoneedle. Moreover, the second end of the polynucleotide can comprise an oligonucleotide capable of hybridizing with a payload (see discussion above related to FIGS. 1a-1d). The system can further comprise a plurality of wells 320, wherein each well can be configured to house a cell 330 to be penetrated by a respective one of the plurality of nanoneedles. The system can further comprise an injecting device 340 that can be configured to move the plurality of nanoneedles to within a defined range of the plurality of wells. In various embodiments, the system may comprise a single nanoneedle and a single well. The nanoneedle, or plurality of nanoneedles, can be housed in or on the injecting device 340. For example, as illustrated in FIG. 3, the plurality of nanoneedles 300 are provided on injecting device 340.

As discussed above, in all of the embodiments of methods, chips and systems provided herein, all features discussed herein related to, for example, the nanoneedle and associated materials, well features and well types, chip features and chip types, polynucleotides and corresponding attachment and coupling features, nanoneedle coatings, aptamers, oligonucleotides, payload and payload types and payload delivery techniques to target (e.g., cell), cell features and cell type, scalable systems, and agricultural applications, apply to any and all embodiments described herein (including, for example, any and all methods, chips and systems embodiments provided herein).

Although specific embodiments and applications of the invention have been described in this specification, these embodiments and applications are exemplary only, and many variations are possible.

### Recitation of Embodiments

Embodiment 1. A method of delivering a payload to a cell comprising providing a nanoneedle, and a polynucleotide, wherein a first end of the polynucleotide comprises an aptamer capable of binding a molecule endogenous to a cell, wherein the first end of the polynucleotide is conjugated to the nanoneedle, and wherein a second end of the polynucleotide comprises an oligonucleotide capable of hybridizing with a payload; contacting the payload with the polynucleotide, wherein the payload contains a nucleotide sequence that is complementary to the oligonucleotide sequence; and inserting the nanoneedle into a cell, wherein upon insertion of the nanoneedle into the cell the payload is released from the polynucleotide.

Embodiment 2. The method of embodiment 1, wherein the nanoneedle is coated with gold atoms.

Embodiment 3. The method of embodiment 2, wherein a thiol group is attached to the first end of the polynucleotide, wherein the polynucleotide is thiolated to the gold atom coated nanoneedle.

Embodiment 4. The method of any of the preceding embodiments, wherein the aptamer is capable of binding to adenosine triphosphate.

Embodiment 5. The method of any of the preceding embodiments, wherein the payload is a nucleotide-based molecule.

Embodiment 6. The method of embodiment 5, wherein the payload comprises DNA.

Embodiment 7. The method of embodiment 5, wherein the payload comprises RNA.

Embodiment 8. The method of embodiment 5, wherein the payload comprises genetic material capable of stably integrating into the genome of the cell.

Embodiment 9. The method of embodiment 5, wherein the payload comprises genetic material capable of transiently expressing a desired target protein.

Embodiment 10. The method of any of the preceding embodiments, wherein the payload inhibits expression of a target gene.

Embodiment 11. The method of embodiment 10, wherein the payload is comprised of siRNA.

Embodiment 12. The method of any of the preceding embodiments, wherein the payload is capable of functionally inhibiting a protein.

Embodiment 13. The method of any of the preceding embodiments, wherein the payload comprises genetic material that expresses a protein capable of functionally inhibiting a desired target.

Embodiment 14. The method of any of embodiments 1 to 13, wherein the payload is circular.

Embodiment 15. The method of any of embodiments 1 to 13, wherein the payload is a linear nucleotide sequence.

Embodiment 16. The method of any of embodiments 1 to 13, wherein the payload is single stranded.

Embodiment 17. The method of embodiment 7, wherein the payload is a RNA plasmid.

Embodiment 18. The method of any one of embodiments 1 to 4, wherein the payload is a protein.

Embodiment 19. The method of any of any one of embodiments 1 to 4, wherein the payload is a small molecule.

Embodiment 20. The method of embodiment 18 or 19, wherein the payload is conjugated to a unique nucleotide sequence capable of hybridizing with the oligonucleotide.

Embodiment 21. A method of delivering a payload to a cell comprising providing a nanoneedle, and a polynucleotide, wherein a first end of the polynucleotide comprises an aptamer capable of binding a molecule endogenous to a cell, wherein the first end of the polynucleotide is conjugated to the nanoneedle, and wherein a second end of the polynucleotide comprises an oligonucleotide capable of hybridizing with a payload; orienting the nanoneedle such that it is configured to be placed into contact with a payload; and inserting the nanoneedle into the inner volume of a well that is configured to house a cell wherein the nanoneedle is configured such that, upon insertion of the nanoneedle into the cell the payload is released from the polynucleotide.

Embodiment 22. The method of embodiment 21, wherein the nanoneedle is coated with gold atoms.

Embodiment 23. The method of embodiment 22, wherein a thiol group is attached to the first end of the polynucleotide, wherein the polynucleotide is thiolated to the gold atom coated nanoneedle.

Embodiment 24. The method of any of one of embodiments 21 to 23, wherein the aptamer is capable of binding to adenosine triphosphate.

Embodiment 25. The method of any of one of embodiments 21 to 24, wherein the payload is a nucleotide-based molecule.

Embodiment 26. The method of embodiment 25, wherein the payload is comprised of DNA.

Embodiment 27. The method of embodiment 25, wherein the payload is comprised of RNA.

Embodiment 28. The method of embodiment 25, wherein the payload comprises genetic material capable of stably integrating into the cells genome.

Embodiment 29. The method of embodiment 25, wherein the payload comprises genetic material capable of transiently expressing a desired target protein.

Embodiment 30. The method of any of one of embodiments 21 to 29, wherein the payload inhibits expression of a target gene.

Embodiment 31. The method of embodiment 30, wherein the payload is comprised of siRNA.

Embodiment 32. The method of any of one of embodiments 21 to 31, wherein the payload is capable of functionally inhibiting a protein.

Embodiment 33. The method of any of one of embodiments 21 to 32, wherein the payload comprises genetic material that expresses a protein capable of functionally inhibiting a desired target.

Embodiment 34. The method of any of one of embodiments 21 to 33, wherein the payload is circular.

Embodiment 35. The method of any of one of embodiments 21 to 33, wherein the payload is a linear nucleotide sequence.

Embodiment 36. The method of any of one of embodiments 21 to 33, wherein the payload is single stranded.

Embodiment 37. The method of embodiment 27, wherein the payload an RNA plasmid.

Embodiment 38. The method of any of one of embodiments 21 to 24, wherein the payload is a protein.

Embodiment 39. The method of any of one of embodiments 21 to 24, wherein the payload is a small molecule.

Embodiment 40. The method of embodiment 38 or 39, wherein the payload is conjugated to a unique nucleotide sequence capable of hybridizing with the oligonucleotide.

Embodiment 41. A chip for delivering a payload comprising a plurality of molecules to a cell comprising a plurality of nanoneedles and a plurality of polynucleotides, wherein a first end of the polynucleotide comprises an aptamer capable of binding a molecule endogenous to a cell, wherein the first end of the polynucleotide is conjugated to the nanoneedle, and wherein a second end of the polynucleotide comprises an oligonucleotide capable of hybridizing with one of a plurality of molecules of a payload.

Embodiment 42. The chip of embodiment 41, wherein the chip is comprised of silicon.

Embodiment 43. The chip of embodiment 41, wherein the nanoneedle is coated with gold atoms.

Embodiment 44. The chip of embodiment 43, wherein a thiol group is attached to the first end of the polynucleotide, wherein the polynucleotide is thiolated to the gold atom coated nanoneedle.

Embodiment 45. The chip of any of one of embodiments 41 to 44, wherein the aptamer is capable of binding to adenosine triphosphate.

Embodiment 46. The chip of any of one of embodiments 41 to 45, wherein the payload is a nucleotide-based molecule.

Embodiment 47. The chip of embodiment 46, wherein the payload is comprised of DNA.

Embodiment 48. The chip of embodiment 46, wherein the payload is comprised of RNA.

Embodiment 49. The chip of embodiment 46, wherein the payload comprises genetic material capable of stably integrating into the cells genome.

Embodiment 50. The chip of embodiment 46, wherein the payload comprises genetic material capable of transiently expressing a desired target protein.

Embodiment 51. The chip of any of one of embodiments 41 to 50, wherein the payload inhibits expression of a target gene.

Embodiment 52. The chip of embodiment 51, wherein the payload is comprised of siRNA.

Embodiment 53. The chip of any of one of embodiments 41 to 52, wherein the payload is capable of functionally inhibiting a protein.

Embodiment 54. The chip of any of one of embodiments 41 to 53, wherein the payload comprises genetic material that expresses a protein capable of functionally inhibiting a desired target.

Embodiment 55. The chip of any of one of embodiments 41 to 54, wherein the payload is circular.

Embodiment 56. The chip of any of one of embodiments 41 to 54, wherein the payload is a linear nucleotide sequence.

Embodiment 57. The chip of any of one of embodiments 41 to 54, wherein the payload is single stranded.

Embodiment 58. The chip of embodiment 48, wherein the payload an RNA plasmid.

Embodiment 59. The chip of any of one of embodiments 41 to 45, wherein the payload is a protein.

Embodiment 60. The method any of one of embodiments 41 to 45, wherein the payload is a small molecule.

Embodiment 61. The method any of one of embodiments 59 and 60, wherein the payload is conjugated to a unique nucleotide sequence capable of hybridizing with the oligonucleotide.

Embodiment 62. A system for delivering a payload comprising of a plurality of molecules to a cell comprising a plurality of nanoneedles, and a plurality of polynucleotides, wherein a first end of a respective polynucleotide of a respective one of the plurality of polynucleotides comprises an aptamer capable of binding a molecule endogenous to the cell, wherein the first end of the respective polynucleotide is conjugated to one of the plurality of nanoneedles, and wherein a second end of the respective polynucleotide comprises an oligonucleotide capable of hybridizing with one of a plurality of molecules of a payload; a plurality of wells; and an injecting device configured to move the plurality of nanoneedles to within the plurality of wells.

Embodiment 63. The system of embodiment 62, wherein the nanoneedle is coated with gold atoms.

Embodiment 64. The system of embodiment 63, wherein a thiol group is attached to the 5′ end of the polynucleotide, wherein the polynucleotide is thiolated to the gold atom coated nanoneedle.

Embodiment 65. The system of any of one of embodiments 62 to 64, wherein the aptamer is capable of binding to adenosine triphosphate.

Embodiment 66. The system of any of one of embodiments 62 to 65, wherein the payload is a nucleotide based molecule.

Embodiment 67. The system of embodiment 66, wherein the payload is comprised of DNA.

Embodiment 68. The system of embodiment 66, wherein the payload is comprised of RNA.

Embodiment 69. The system of embodiment 66, wherein the payload comprises genetic material capable of stably integrating into the cells genome.

Embodiment 70. The system of embodiment 66, wherein the payload comprises genetic material capable of transiently expressing a desired target protein.

Embodiment 71. The system of any of one of embodiments 62 to 70, wherein the payload inhibits expression of a target gene.

Embodiment 72. The system of embodiment 71, wherein the payload is comprised of siRNA.

Embodiment 73. The system of any of one of embodiments 62 to 72, wherein the payload is capable of functionally inhibiting a protein.

Embodiment 74. The system of any of one of embodiments 62 to 73, wherein the payload comprises genetic material that expresses a protein capable of functionally inhibiting a desired target.

Embodiment 75. The system of any of one of embodiments 62 to 74, wherein the payload is circular.

Embodiment 76. The system of any of one of embodiments 62 to 74, wherein the payload is a linear nucleotide sequence.

Embodiment 77. The system of any of one of embodiments 62 to 74, wherein the payload is single stranded.

Embodiment 78. The system of embodiment 68, wherein the payload an RNA plasmid.

Embodiment 79. The system of any of one of embodiments 62 to 65, wherein the payload is a protein.

Embodiment 80. The system of any of one of embodiments 62 to 65, wherein the payload is a small molecule.

Embodiment 81. The system of embodiment 79 or 80, wherein the payload is conjugated to a unique nucleotide sequence capable of hybridizing with the oligonucleotide.

Embodiment 82. The system of any of one of embodiments 62 to 81, wherein the each of the plurality of wells is configured to house a cell to be penetrated by a nanoneedle.

Embodiment 83. The system of any of one of embodiments 62 to 82, wherein the injecting device is configured to move the plurality of nanoneedles to within the plurality of wells.

What is claimed:

1. A method of delivering a payload to a cell comprising
a. providing a nanoneedle and a polynucleotide,
wherein a first end of the polynucleotide comprises an aptamer capable of binding a molecule endogenous to a cell,
wherein the first end of the polynucleotide is conjugated to the nanoneedle, and
wherein a second end of the polynucleotide comprises an oligonucleotide capable of hybridizing with a payload;
b. contacting the payload with the polynucleotide, wherein the payload contains a nucleotide sequence that is complementary to the oligonucleotide sequence; and
c. inserting the nanoneedle into a cell, wherein upon insertion of the nanoneedle into the cell the payload is released from the polynucleotide.

2. The method of claim 1 wherein the aptamer is capable of binding to adenosine triphosphate.

3. The method of claim 1 wherein the payload is a nucleotide-based molecule.

4. The method of claim 1, wherein the payload comprises genetic material capable of stably integrating into the genome of the cell.

5. The method of claim 1, wherein the payload comprises genetic material capable of transiently expressing a desired target protein.

6. The method of claim 1, wherein the payload inhibits expression of a target gene.

7. The method of claim 1, wherein the payload is capable of functionally inhibiting a protein.

8. The method of claim 1, wherein the payload comprises genetic material that expresses a protein capable of functionally inhibiting a desired target.

9. The method of claim 1, wherein the payload is a protein.

10. The method of claim 1, wherein the payload is a small molecule.

11. The method of claim 1, wherein the payload is conjugated to a unique nucleotide sequence capable of hybridizing with the oligonucleotide.

12. The method of claim 1, further comprising inserting the nanoneedle into an inner volume of a well that is configured to house the cell, wherein the nanoneedle is configured such that, upon insertion of the nanoneedle into the cell, the payload is released from the polynucleotide.

13. A chip for delivering a payload to a cell, the chip comprising
a solid support, and
a nanoneedle attached to the solid support and configured to receive a polynucleotide,
wherein a first end of the polynucleotide comprises an aptamer capable of binding a molecule endogenous to a cell,
wherein the first end of the polynucleotide is capable of conjugating to the nanoneedle, and
wherein a second end of the polynucleotide comprises an oligonucleotide capable of hybridizing with one of a plurality of molecules of a payload.

14. The chip of claim 13, wherein the chip is comprised of silicon.

15. The chip of claim 13, wherein the aptamer is capable of binding to adenosine triphosphate.

16. The chip of claim 13, wherein the payload is a nucleotide-based molecule.

17. The chip of claim 13, wherein the payload comprises genetic material capable of stably integrating into the cells genome.

18. The chip of claim 13, wherein the payload comprises genetic material capable of transiently expressing a desired target protein.

19. The chip of claim 13, wherein the payload inhibits expression of a target gene.

20. The chip of claim 13, wherein the payload is capable of functionally inhibiting a protein.

21. The chip of claim 13, wherein the payload comprises genetic material that expresses a protein capable of functionally inhibiting a desired target.

22. The chip of claim 13, wherein the payload is a protein.

23. The method of claim 13, wherein the payload is a small molecule.

24. The method of claim 13, wherein the payload is conjugated to a unique nucleotide sequence capable of hybridizing with the oligonucleotide.

25. The method of claim 13, further comprising a plurality of nanoneedles attached to the solid support, wherein each of the plurality of nanoneedles is configured to receive a polynucleotide.

26. A system for delivering a payload comprising of a plurality of molecules to a cell comprising
a. a plurality of nanoneedles, and a plurality of polynucleotides,
wherein a first end of a respective polynucleotide of a respective one of the plurality of polynucleotides comprises an aptamer capable of binding a molecule endogenous to the cell,
wherein the first end of the respective polynucleotide is conjugated to one of the plurality of nanoneedles, and
wherein a second end of the respective polynucleotide comprises an oligonucleotide capable of hybridizing with one of a plurality of molecules of a payload;
b. a plurality of wells; and
c. an injecting device that houses the plurality of nanoneedles thereon, and is configured to move the plurality of nanoneedles to within the plurality of wells.

27. The system of claim 25, wherein the aptamer is capable of binding to adenosine triphosphate.

28. The system of claim 25, wherein the payload is a nucleotide based molecule.

29. The system of claim 25, wherein the payload comprises genetic material capable of stably integrating into the cells genome.

30. The system of claim 25, wherein the payload comprises genetic material capable of transiently expressing a desired target protein.

31. The system of claim 25, wherein the payload inhibits expression of a target gene.

32. The system of claim 25, wherein the payload is capable of functionally inhibiting a protein.

33. The system of claim 35, wherein the payload comprises genetic material that expresses a protein capable of functionally inhibiting a desired target.

34. The system of claim 25, wherein the payload is a protein.

35. The system of claim 25, wherein the payload is a small molecule.

36. The system of claim 25, wherein the payload is conjugated to a unique nucleotide sequence capable of hybridizing with the oligonucleotide.

37. The system of claim 25, wherein the each of the plurality of wells is configured to house a cell to be penetrated by a nanoneedle.

38. The system of claim 25, wherein the injecting device is configured to move the plurality of nanoneedles to within the plurality of wells.
